(19) United States
(12) Patent Application Publication
(10) Pub. No.: US 2015/0003591 A1
(43) Pub. Date: Jan. 1, 2015

(54) NUCLEAR IMAGING SYSTEM

(71) Applicant: KONINKLIJKE PHILIPS N.V., EINDHOVEN (NL)

(72) Inventors: Bernd Schweizer, Ketsch (DE); Heinrich Johannes Eckhard Von Busc, Erlangen (DE); Carolina Ribbing, Aachen (DE); Andreas Goedicke, Aachen (DE)

(21) Appl. No.: 14/371,286

(22) PCT Filed: Jan. 18, 2013
(86) PCT No.: PCT/IB2013/050483

§ 371 Date: Jul. 9, 2014

Related U.S. Application Data
(60) Provisional application No. 61/589,900, filed on Jan. 24, 2012.

(51) Int. Cl. A61B 6/00; G01R 33/48; A61B 6/03

(52) U.S. Cl. CPC A61B 6/4417; A61B 6/037; A61B 6/5247; G01R 33/481; A61B 6/481
USPC 378/62

(57) ABSTRACT

Nuclear Imaging System The invention relates to a nuclear imaging system (1) for imaging an object (3) in an examination region. Multiple x-rays sources (2) generate first radiation being x-ray radiation (5), wherein the x-ray sources are arranged such that the x-ray radiation is indicative of a property of the object. A detection unit (6) detects second radiation (7) from a nuclear element (8), after the second radiation has the traversed the object, and the first radiation generated by the multiple x-ray sources, thereby inherently registering the detection of the first radiation and the second radiation. A reconstruction unit (9) reconstructs a corrected nuclear image of the object based on the detected first radiation and the detected second radiation, wherein the nuclear image is corrected with respect to the property of the object and, because of the inherent registration, does not comprise image artifacts caused by registration errors.

### FIELD OF THE INVENTION

The invention relates to a nuclear imaging system, a nuclear imaging method and a nuclear imaging computer program for imaging an object in an examination region.

### BACKGROUND OF THE INVENTION

US 2010/0331665 A1 discloses an apparatus for combined magnetic resonance (MR) tomography and positron emission tomography (PET) imaging. The apparatus is adapted to record PET image data of a person under examination from an examination area. The apparatus comprises a scanning unit for scanning a prespecified area of the person under examination, wherein a contour of the person is determined based on the scanning. The scanning unit includes one or several x-ray sources for illuminating the person with x-ray radiation and corresponding one or several x-ray detectors for detecting the x-ray radiation after having been backscatterd from the surface of the person, wherein the contour is determined based on the detected backscattered x-ray radiation. The apparatus further comprises a processing unit for carrying out an absorption correction of PET image data, which were previously recorded from the prespecified area of the person under examination, based on the determined contour. The correction of the PET image data based on the contour, which is determined based on the scanning by the scanning unit, is relatively inaccurate such that the PET image data comprise artifacts.

### SUMMARY OF THE INVENTION

It is an object of the present invention to provide a nuclear imaging system, a nuclear imaging method and a nuclear imaging computer program for imaging an object in an examination region, wherein the quality of the nuclear image can be improved.

In a first aspect of the present invention a nuclear imaging system for imaging an object in an examination region is presented, wherein the nuclear imaging system comprises:
multiple x-rays sources for generating first radiation being x-ray radiation, the x-ray sources being arrangable such that the x-ray radiation is indicative of a property of the object,
a detection unit for detecting second radiation from a nuclear element, after the second radiation has traversed the object, and the first radiation generated by the multiple x-ray sources,
a reconstruction unit for reconstructing a corrected nuclear image of the object based on the detected first radiation and the detected second radiation, wherein the nuclear image is corrected with respect to the property of the object.

Since the detection unit detects the second radiation and the first radiation, the detection of the first radiation and the second radiation is automatically registered with respect to each other. A reconstruction of the corrected nuclear image, which considers both, the first radiation and the second radiation, can therefore be performed without registration errors, thereby improving the quality of the corrected nuclear image.

The property of the object, of which the x-ray radiation is indicative, is preferentially the absorption or a movement of the object, wherein the movement may be defined by the positions of the object at different times.

The x-ray sources are preferentially miniaturized x-ray sources.

In an embodiment, the property of the object, of which the x-ray radiation is indicative, is the absorption, wherein the multiple x-ray sources are arranged to allow the generated x-ray radiation to traverse the object. The multiple x-ray sources can be arranged around the examination region for generating first radiation traversing the object in different directions, wherein the detection unit is adapted for detecting the first radiation having traversed the object in different directions. In particular, the multiple x-ray sources can be arranged in a full or partial ring around the examination region. Since the multiple x-ray sources are arranged such that the first radiation traverses the object in different directions, it is not necessary to rotate an x-ray source around the examination region, thereby simplifying the technical construction of the imaging system. The second radiation from the nuclear element can be detected by the detection unit, after the second radiation has completely or partly traversed the object.

In a preferred embodiment the reconstruction unit is adapted to reconstruct an attenuation image of the object being indicative of the absorption distribution within the object based on the detected first radiation and to generate an attenuation corrected nuclear image based on the detected second radiation and the reconstructed attenuation image. In particular, the nuclear element is a PET contrast agent, wherein the detection unit comprises a detector ring surrounding the examination region for detecting the second radiation in different directions, wherein the reconstruction unit is adapted to reconstruct an attenuation corrected PET image based on the detected second radiation and the attenuation image. This further increases the quality of the corrected nuclear image being, in this embodiment, a PET image. The detector ring for detecting the second radiation, i.e. the PET detector ring and a half ring or a full ring of the multiple x-ray sources may be axially offset with respect to each or they may be integrated into each other. The nuclear imaging system can further comprise an MR scanning unit for generating an MR image of the object such that the nuclear imaging system is a PET/MR system with an additional ring of x-ray sources.

In another embodiment, the multiple x-ray sources are adapted to be arranged on the object such that the x-ray radiation is indicative of a movement of the object. The reconstruction unit can then be adapted to determine the movement of the object based on the detected first radiation and to reconstruct a motion corrected nuclear image based on the detected second radiation and the determined movement of the object. In this embodiment, the nuclear element is preferentially a nuclear single photon emission tomography (SPECT) contrast agent, wherein the detection unit comprises at least one gamma camera being adapted to detect the second radiation in different directions and to detect the first radiation, wherein the reconstruction unit is adapted to reconstruct a motion corrected SPECT image based on the second radiation detected in the different directions and the determined movement of the object. This allows generating a motion corrected SPECT image having reduced motion artifacts or no motion artifacts at all.

The reconstruction unit is preferentially adapted to detect the position of the respective x-ray source within a gamma camera image, for instance, by thresholding or by using other segmentation techniques, wherein based on the positions of the x-ray sources within the gamma camera images the movement of the object can be determined in a reference coordinate system defined by the gamma camera. Since also the detected second radiation forming nuclear data is acquired by the gamma camera, also the nuclear data are known with respect to the reference coordinate system defined by the gamma camera. The positions of the x-ray sources in the gamma camera images can therefore easily be used for reconstructing a motion corrected SPECT image, without requiring a registration of the nuclear data with the detected positions of the x-ray sources.

It is further preferred that the at least one gamma camera is adapted to detect also the first radiation in different directions, wherein the reconstruction unit is adapted to determine the positions of the multiple x-ray sources over time from the first radiation detected in different directions, thereby determining the movement of the object. For instance, a computed tomography reconstruction technique can be used for determining the positions of the multiple x-ray sources over time. This allows accurately determining the positions of the multiple x-ray sources over time and, thus, precisely the movement of the object.

It is further preferred that the x-rays sources are adapted to be activated in a predefined temporal pattern, wherein the detection unit is adapted to detect the first radiation based on the predefined temporal pattern. For instance, the detection unit can be adapted to determine to which x-ray source which detected first radiation corresponds based on the predefined temporal pattern. In an embodiment, in accordance with the temporal pattern at a time only one x-ray source is activated. Moreover, the x-ray sources can be adapted to generate x-ray radiation having an intensity being modulated in accordance with modulation characteristics, wherein the detection unit can be adapted to separate the first radiation from the second radiation based on the modulation characteristics. In particular, the intensity of different x-ray sources can be modulated differently in accordance with different modulation characteristics, wherein the detection unit is adapted to separate the first radiation from the different x-ray sources based on the different modulation characteristics. Furthermore, the detection unit can be adapted to detect the first radiation in a first energy range and the second radiation in a second energy range, in order to separate these detected radiations from each other. These techniques allow detecting different kinds of radiation by using the same detection unit.

In a further aspect of the present invention a nuclear imaging method for imaging an object in an examination region is presented, wherein the nuclear imaging method comprises:
generating first radiation being x-ray radiation by multiple x-ray sources, the x-ray sources being arrangable such that the x-ray radiation is indicative of a property of the object,
detecting second radiation from a nuclear element by a detection unit, after the radiation has traversed the object, and the first radiation generated by the multiple x-ray sources,
reconstructing a corrected nuclear image of the object based on the detected first radiation and the detected second radiation by a reconstruction unit, wherein the nuclear image is corrected with respect to the property of the object.

The first radiation can be detected before, after or simultaneously with detecting the second radiation.

In a further aspect of the present invention a nuclear imaging computer program for imaging an object is presented, wherein the nuclear imaging computer program comprises program code means for causing a nuclear imaging system as defined in claim 1 to carry out the steps of the nuclear imaging method as defined in claim 14, when the nuclear imaging computer program is run on a computer controlling the nuclear imaging system.

It shall be understood that the nuclear imaging system of claim 1, the nuclear imaging method of claim 14, and the nuclear imaging computer program of claim 15 have similar and/or identical preferred embodiments as defined in the dependent claims.

It shall be understood that a preferred embodiment of the invention can also be any combination of the dependent claims with the respective independent claim.

These and other aspects of the invention will be apparent from and elucidated with reference to the embodiment(s) described hereinafter.

### BRIEF DESCRIPTION OF THE DRAWINGS

In the following drawings:

FIG. 1 shows schematically and exemplarily an embodiment of a first nuclear imaging system being a PET/MR imaging system,

FIG. 2 shows schematically and exemplarily x-ray sources of the PET/MR imaging system,

FIG. 3 shows schematically and exemplarily an embodiment of a second nuclear imaging system being a SPECT imaging system,

FIG. 4 shows exemplarily a spectrum of a SPECT contrast agent acquired by a gamma camera of the SPECT imaging system, and

FIG. 5 shows a flowchart exemplarily illustrating an embodiment of a nuclear imaging method for imaging an object in an examination region.

### DETAILED DESCRIPTION OF EMBODIMENTS

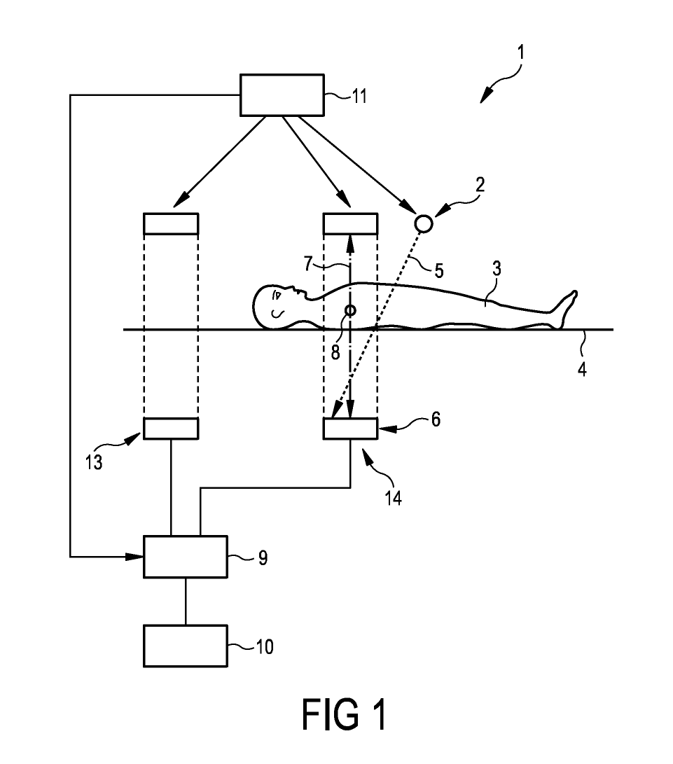

FIG. 1 shows schematically and exemplarily an embodiment of a nuclear imaging system for imaging an object in an examination region. The nuclear imaging system 1 is, in this embodiment, a PET/MR imaging system.

The nuclear imaging system 1 comprises multiple x-ray sources 2 for generating first radiation 5 being x-ray radiation. The x-ray sources 2 are arranged such that the x-ray radiation 5 is indicative of a property of the object 3 being, in this embodiment, a person 3 lying on a table 4. The nuclear imaging system 1 further comprises a detection unit 6 for detecting second radiation 7 from a nuclear element 8 within the object 3, after the second radiation 7 has traversed the object 3, and the first radiation 5 generated by the multiple x-ray sources 2. A reconstruction unit 9 reconstructs a corrected nuclear image of the object 3 based on the detected first radiation 5 and the detected second radiation 7, wherein the nuclear image is corrected with respect to the property of the object 3. The reconstructed attenuation-corrected PET image is finally shown on a display 10.

The nuclear imaging system 1 further comprises an MR signals acquisition unit 13 for acquiring MR signals, which are provided to the reconstruction unit 9 for reconstructing an MR image.

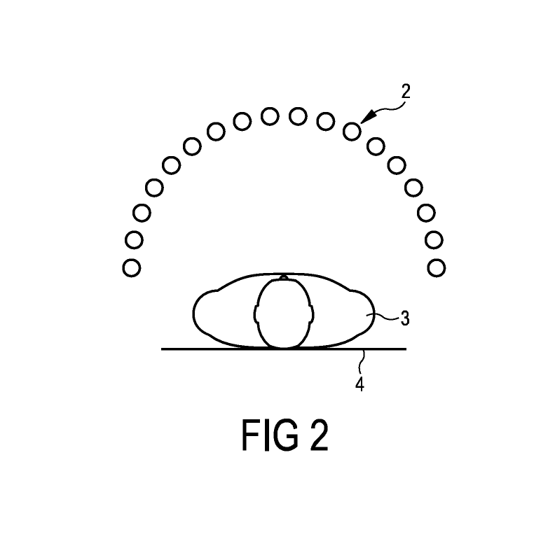

The x-ray sources 2 are miniature x-ray sources, which are arranged around the examination region for generating the first radiation 5 traversing the object 3 in different directions, wherein the detection unit 6 is adapted to detect the first radiation having traversed the object 3 in different directions. In this embodiment, the x-ray sources 2 are arranged in a half ring around the examination region comprising the object 3 on the table 4. The half ring of x-ray sources 2 is arranged in a plane being perpendicular to the longitudinal axis of the table 4 and of the person 3 as schematically and exemplarily shown in FIG. 2. The arrangement of the x-ray sources 2 constitutes a low level computed tomography unit integrated in the PET/MR imaging system, which allows generation of attenuation maps.

The reconstruction unit 9 is adapted to reconstruct an attenuation image of the object 3 being indicative of the absorption distribution within the object 3 based on the detected first radiation 5 and to generate an attenuation corrected nuclear image based on the detected second radiation 7 and the reconstructed attenuation image. In this embodiment, the nuclear element is a PET contrast agent, wherein the detection unit 6 is a PET detector ring surrounding the examination region for detecting the second radiation 7 in different directions, wherein the reconstruction unit 9 is adapted to reconstruct an attenuation corrected PET image based on the detected second radiation 7 and the attenuation image.

The reconstruction unit 9 can be adapted to use existing fan-beam or cone-beam reconstruction algorithms known from the field of computed tomography for reconstructing a low-level tomography image which can be regarded as being an x-ray transmission map. The reconstruction unit 9 considers the oblique ray angle with respect to a plane transversal to the scanner's axis. For example, known versions of the Feldkamp algorithm, which consider oblique ray angles, like the algorithm disclosed in the article “Cone-beam volume CT breast imaging: Feasibility study” by B. Chen et al., Medical Physics, volume 29, number 5, pages 755 to 770 (2002)”, which is herewith incorporated by reference, can be used by the reconstruction unit 9. The reconstruction 9 can also be adapted to use iterative reconstruction algorithms for reconstructing the low-level tomography image. For instance, the system matrix of the acquisition geometry can be calculated, thereby transforming the reconstruction problem in a system of linear equations, which can be solved by the reconstruction unit by known iterative algorithms like maximum-likelihood expectation-maximization (MLEM) or algebraic reconstruction technique (ART) algorithms.

The low-level computed tomography image is an x-ray attenuation image, which is transformed by the reconstruction unit 9 into attenuation values for PET photons having an energy of about 511 keV. This transformation of the x-ray attenuation map to attenuation values for 511 keV can be performed by using transformations, which are known from the PET/CT field, for instance, by means of a known bi-linear transformation from Hounsfield units to the attenuation values for 511 keV. The resulting attenuation map for 511 keV photons is used together with the detected second radiation from the PET contrast agent 8 by the reconstruction unit 9 for generating the corrected PET image by using known reconstruction and correction methods. For instance, the reconstruction and correction methods disclosed in the book “Positron Emission Tomography—Clinical Practice” by P. E. Valk et al., Springer-Verlag London Limited, pages 12 to 13 (2006), the article “Systematic and Distributed Time-of-Flight List Mode PET Reconstruction” by W. Wang et al., Nuclear Science Symposium Conference Record, volume 3, pages 1715 to 1722 (2006) and the article “Application of the row action maximum likelihood algorithm with spherical basis functions to clinical PET imaging” by M. E. Daube-Witherspoon et al., Nuclear Science, volume 48, pages 24 to 30 (2001) can be used, which are herewith incorporated by reference.

The nuclear imaging system 1 further comprises a controller 11 for controlling the MR signals acquisition unit 13, a PET part 14 of the imaging system 1 comprising at least the PET detector ring, the x-ray sources 2 and the reconstruction unit 9.

The number of x-ray sources 2 can be relatively large, for instance, the nuclear imaging system 1 can comprise a number of x-ray sources between 5 and 100. They can be arranged in a half ring as schematically and exemplarily shown in FIG. 2, or they can be arranged in a full ring or in selected sections of a full ring. The ring is preferentially axially offset with respect to the PET detector ring 6.

The arrangement of x-ray sources 2 does not need to be rotated like in a conventional x-ray computed tomography scanner, but at every time point only one x-ray source 2 can be activated such that in the combination of the respective given x-ray source and the PET detector element the respective x-ray path through the person 3 is well defined and can be used for reconstructing a computed tomography image by using known computed tomography reconstruction algorithms like a filtered back projection algorithm or a Radon inversion algorithm. The x-ray sources 2 can also be activated in another predefined temporal pattern, wherein the detection unit 6 can be adapted to detect the x-ray radiation based on the predefined temporal pattern.

The x-ray sources 2 can also be adapted to generate x-ray radiation having an intensity being modulated in accordance with modulation characteristics, wherein the detection unit 6 can be adapted to separate the first radiation from the second radiation based on the modulation characteristics. Moreover, the intensity of different x-ray sources can be modulated differently in accordance with different modulation characteristics, wherein the detection unit 6 can be adapted to separate the x-ray radiation from the different x-ray sources based on the different modulation characteristics. For detecting x-ray radiation of a certain x-ray source based on the modulation characteristics the detection unit can use a lock-in technique or a Fourier transformation. The modulation characteristics can be defined, for instance, by the modulation frequency. For example, different x-ray sources 2 can be modulated with different modulation frequencies, wherein the detection unit 6 can be adapted to separate x-ray radiation originating from different x-ray sources based on the respective frequency. For instance, the detection unit 6 can be adapted to generate a detection signal based on the detected first and second radiation, to Fourier transform the detection signal and to determine which frequency component of the Fourier transformed detection signal corresponds to which x-ray source based on the modulation frequencies with which the respective x-ray sources are operated. The intensity can be modulated by, for instance, switching the x-ray sources on and off, wherein different x-ray sources are operated with different switching frequencies, in order to allow the detection unit to separate the different contributions to the acquired detection signal from the different x-ray sources. The switching frequency is, in this example, the modulation frequency.

Thus, the emission strengths of each miniature x-ray source can be temporally modulated such that with a corresponding detection principle like a lock-in technique or a technique using a Fourier transformation the x-ray transmission signal, i.e. the part of the detection signal being indicative of the transmitted x-ray radiation, can be clearly separated from scattered photons due to positron anihilition of the PET contrast agent 8 within the person 3, and also x-ray transmission signals which correspond to different x-ray sources can be clearly separated from each other.

The detection unit 6 can also be adapted to detect the first radiation in a first energy range and the second radiation in a second energy range, in order to separate the first radiation from the second radiation. In particular, the detection of transmitted x-ray photons can be performed in an energy window well below a PET signal window around 511 keV. The x-ray radiation, i.e. the first radiation, preferentially has an energy between about 30 to about 120 keV. The first energy range covers therefore preferentially this energy range from about 30 to about 120 keV, wherein the second energy range includes preferentially 511 keV. The first energy range of about 30 to about 120 keV is high enough to provide enough transmission through body tissue and is still in an operational range of miniature x-ray sources.

The above described separation techniques allow the x-ray sources to emit the x-ray radiation simultaneously, while the detector signal generated by the detection unit can still be de-multiplexed.

The miniature x-ray sources 2 are preferentially electron impact sources. In an embodiment, the x-ray sources 2 can have a pyroelectric cathode, making high-voltage cables obsolete. They can be housed in a standard TO8 package having, for example, a diameter of 15 mm and a height of 10 mm and powered by a standard 9 V battery. Their transmission anode can be a copper target on a beryllium window. The photon flux can be pulsed according to the heat, wherein a cooling cycle of the cathode can be provided with a cycling time of, for instance, 3 min. In the described PET/MR system the respective x-ray source can also be arranged in a larger housing of, for instance 185 mm×35 mm. Moreover, the x-ray sources can also have a transmission anode with a silver, tungsten or gold target. In an embodiment, the x-ray sources are x-ray sources of the company Amptek named “Cool-X” or “Mini-X”, of the company Oxford instruments named “Eclipse”, or of the company Xoft/iCAD. Also other x-ray sources could be used. For instance, a centimeter sized x-ray source incorporating a pyroelectric cathode, which is, for instance, based on lithium niobate and which operates at about 100 keV, could be used by the nuclear imaging system 1. Yet other possible x-ray sources are triboluminiscens sources emitting in the x-ray range and miniature x-ray sources which are not of electron-impact type, such as x-ray emitting laser plasma sources.

In known PET/MR imaging systems the generation of PET attenuation maps out of MR images is a real challenge. The MR intensities do not bear physical resemblance to photon attenuation coefficients, but show a signal which is connected to the proton density of the material. Ray value mapping does not work, since, for instance, bone and air both appear black in MR images, whereas they are drastically different with respect to PET photon attenuation. A PET image, which is corrected depending on an MR based attenuation map, comprises therefore artifacts, which are caused by the MR-based attenuation correction.

In known sequential PET/MR imaging systems an MR image, which serves as a basis for an attenuation map, is acquired approximately 10 to 20 minutes before the acquisition of the PET imaging data. This results in a possible geometric mismatch between the PET imaging data and the attenuation map due to patient motion before or during the PET scan. In contrast, the nuclear imaging system 1 described above with reference to FIG. 1 allows acquiring one or several attenuation maps during the PET scan. This can result in a better image quality and quantification of the reconstructed PET image.

Moreover, known PET/MR imaging systems use algorithms for extracting attenuation information from MR images by making assumptions about the geometry of the person or the image content. These assumptions may not hold for previously operated persons with changed anatomy or animal subjects for pre-clinical studies. In contrast, the reconstruction unit 9 described above with reference to FIG. 1 preferentially does not make these assumptions, but records the true distribution of attenuating material by using the detected first radiation, i.e. by using the detected transmitted x-ray radiation.

Furthermore, metal implants like dental fillings, hip replacements, pacemakers, implanted ports for chemotherapy, et cetera impose severe imaging problems for MR scanners. In particular, in an MR image there is either a strong distortion or even just missing information, i.e. a “hole” in the person seems to be visible in the MR image, which is transferred to the attenuation map used for generating an attenuation corrected PET image. In contrast, the attenuation map generated by the nuclear imaging system 1 described above with reference to FIG. 1 can generate a low-level computed tomography image, wherein known metal artifact reduction algorithms, which are known from the x-ray CT field, can be used for reducing metal artifacts. The generation of the corrected PET image can then be based on this metal-artifact-corrected low-level computed tomography image, thereby reducing artifacts in the corrected PET image, which may be caused by metal implants.

In addition, in known PET/MR imaging systems the transversal field of view radius of the MR signals acquisition unit is generally smaller than the field of view radius of the PET acquisition unit. This can lead to truncated MR information, for instance, parts of the arms of a person can be missing, which leads to a truncated attenuation map, whereas the nuclear imaging system 1 described above with reference to FIG. 1 allows generating a non-truncated attenuation map based on the detected first radiation.

Moreover, in known PET/MR imaging systems MR image information may be geometrically distorted, which may lead to inconsistent attenuation maps. The nuclear imaging system 1 described above with reference to FIG. 1 allows generating geometrically accurate attenuation maps based on the detected first radiation, which leads to an improved quality of the corrected PET image. Moreover, the reconstruction unit can also be adapted to correct the geometrical distortions of the MR information based on the low-level computed tomography image.

The geometrical distortions, which can be corrected by using the low-level computed tomography image, can be, for instance, caused by the limited field of view of the MR imaging system. For instance, parts of a person like arms may not be shown on the MR image because of the limited field of view. In an embodiment, for performing the correction the contour of a person is extracted in an MR image and compared with a corresponding contour in the low-level computed tomography image. If deviations between these two contours are larger than a predefined threshold, corresponding image regions in the MR image can be filled with image information from the low-level computed tomography image. More details of this known correction technique are disclosed, for example, in the article “MR-based Attenuation Correction for a Whole-body Sequential PET/MR System” by Z. Hu et at., IEEE Nuclear Science Symposium Medical Imaging Conference, pages 2119-2122 (2010), which is herewith incorporated by reference. Geometrical distortions can also be caused by metallic elements like metal implants within the person. The metal implants lead to metal artifacts, which are visible in the MR images as relatively large black regions. These black regions can be filled with image information from corresponding regions in the low-level computed tomography image.

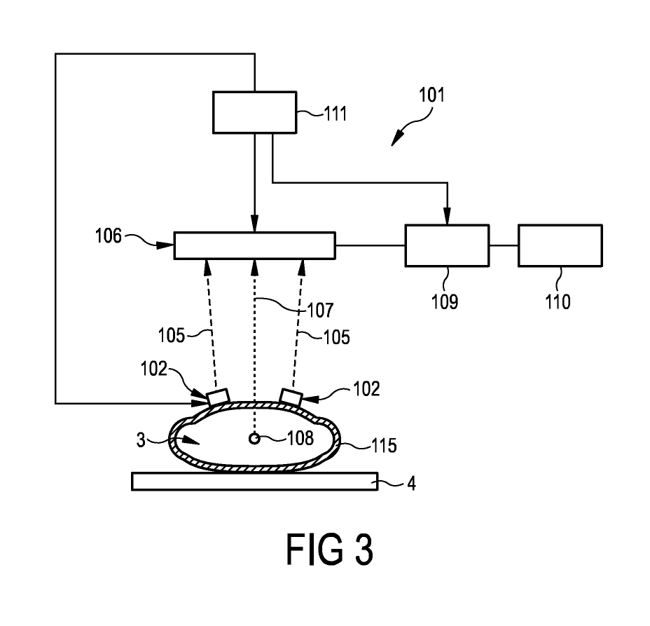

FIG. 3 shows schematically and exemplarily a further embodiment of a nuclear imaging system for imaging an object in an examination region. The nuclear imaging system 101 shown in FIG. 3 comprises multiple x-ray sources 102 for generating first radiation 105 being x-ray radiation, wherein the x-ray sources 102 are arranged such that the x-ray radiation 105 is indicative of a property of the object 3. In this embodiment, the x-ray sources 102 are arranged on a person 3 lying on a table 4 such that the x-ray radiation 105 is indicative of a movement of the person 3. Thus, in this embodiment the property of the object is not the attenuation as in the other embodiment described above with reference to FIGS. 1 and 2, but the property, of which the x-ray radiation is indicative, is the movement of the object, wherein the movement can be defined by the position of the object at different times.

The nuclear imaging system 101 is a SPECT imaging system comprising a detection unit 106 for detecting second radiation 107 from a nuclear element 108, after the second radiation 107 has traversed the object 3, and the first radiation 105, i.e. the x-ray radiation, generated by the multiple x-ray sources 102. In this embodiment, the nuclear element 108 is a SPECT contrast agent, wherein the detection unit 106 comprises at least one gamma camera being adapted to detect the nuclear radiation 107, i.e. the second radiation from the SPECT contrast agent 108, in different directions and to detect the first radiation 105 preferentially also in different directions. In particular, the at least one gamma camera can be mounted on a rotating gantry (not shown in FIG. 3) for allowing the at least one gamma camera to detect the first and second radiations 105, 107 in different directions.

The nuclear imaging system 101 further comprises a reconstruction unit 109 for reconstructing a corrected nuclear image of the object 3 based on the detected first radiation 105 and the detected second radiation 107, wherein the nuclear image is corrected with respect to the property of the object 3 being, in this embodiment, a movement of the person 3. In particular, the reconstruction unit 109 is adapted to determine the movement of the person 3 based on the detected first radiation 105 and to reconstruct a motion corrected nuclear image based on the detected second radiation 107 and the determined movement of the person 3. Preferentially, the reconstruction unit 109 is adapted to determine the positions of the multiple x-ray sources 102 over time from the first radiation 105 detected in different directions, in order to determine the movement of the person 3. The nuclear image can be shown on a display 110.

The x-ray sources 102 can be mounted on a thin metal foil, which may be a lead, tungsten or molybdenum foil and which is provided on a belt 115. If in another embodiment instead of the belt 115 another means like an adhesive is used for arranging the x-ray sources 102 on the person 3, the thin metal can be arranged on other means for attaching the x-ray sources 102. The thickness and the material of the metal foil are chosen such that the radiation from the x-ray sources is at least partially blocked, without substantially blocking the second, nuclear radiation. In this embodiment the first radiation is within an energy range of 3 to 50 keV and the second radiation has an energy of about 140 keV. Thus, the material and the thickness of the metal foil are preferentially adapted such that radiation having an energy of about 140 keV transmits through the metal foil and radiation within the energy range from 3 to 50 keV is mostly blocked by the metal foil. In another embodiment, other attaching means can be used for attaching the x-ray sources to the person like attaching means known from attaching electrocardiogram electrodes to a body surface.

The x-ray sources 102 can be switched on and off, even for small fractions of a single SPECT frame duration. This switching can be used to separate the second radiation, i.e. the original SPECT image, from the tracking information, i.e. from the first radiation, by differential techniques like a simple subtraction technique or by a lock-in technique. In a preferred embodiment, the signal-to-background ratio is increased by operating the x-ray sources 102 in the low-energy tail of the original SPECT scatter spectrum, i.e. by using first radiation 105 being in a low-energy tale of the second radiation 107.

The x-ray sources 102 can be miniature x-ray sources, in particular, electron impact sources with filament, field emitting, ferroelectric or pyroelectric cathodes. They have preferentially a diameter in the millimeter to centimeter range and a correspondingly small x-ray focus. They provide sufficient intensity to produce a clearly visible signal at least in a low-energy region of the detected second radiation 107, in particular, of the SPECT spectrum. In an embodiment, the x-ray sources are x-ray sources of the company Moxtek named Magnum. However, also other x-ray sources can be used like x-ray sources of the Axxent type from the company iCAD.

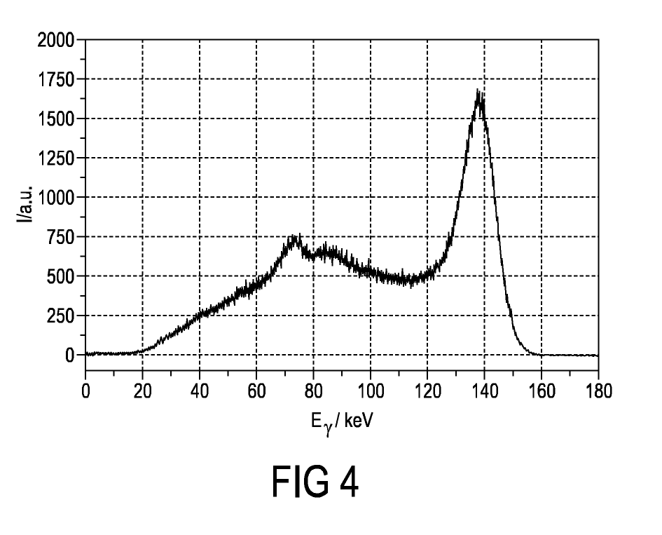

The SPECT contrast agent 108 is, for instance, Tc-99m. However, also other SPECT contrast agents can be used with the nuclear imaging system shown in FIG. 3. The gamma camera is, for instance, based on sodium iodide, wherein a spectrum of Tc-99n recorded by such a gamma camera is schematically and exemplarily shown in FIG. 4, which illustrates the intensity of the detected second radiation depending on the energy.

In FIG. 4 a signal peak around 140 keV as well as a broad region of scatter is clearly visible. In order to achieve a high signal-to-background ratio of the photons emitted by the x-ray sources 102, the first radiation is preferentially in a spectral region of low scatter content. In this embodiment the x-ray sources 102 are therefore operated to give an energy range between 3 and 50 keV.

The reconstruction unit 109 is preferentially adapted to detect the positions of the x-ray sources within gamma camera images, which are acquired by the at least one gamma camera of the detection unit 106 over time, wherein these detected positions of the x-ray sources within the gamma camera images are used by the reconstruction unit 109 for determining the movement of the x-ray sources 102 and, thus, the motion of the person 3. The x-ray sources within the gamma camera images can be detected by known segmentation techniques, which may be based on thresholding, and the reconstruction unit 109 can perform a tomographic reconstruction of the positions of the x-ray sources 102 over time for determining the motion. Specifically, each x-ray source 102 may approximate a point-like photon source, wherein in an embodiment two or more gamma cameras are used, in order to allow the reconstruction unit 109 to determine the center positions of the x-ray sources 102 from a single measurement.

For determining the position of an x-ray source standard three-dimensional reconstruction techniques which are known, for instance, from optical tracking systems can be used. Alternatively, the collimation can be used to determine the position of the respective x-ray source. For instance, the barycenter of the detection events caused by the respective x-ray source can be determined, wherein it can be assumed that the respective x-ray source is approximately located on a line, which is perpendicular to the detection surface of the detection unit and which intersects the detection surface at the determined barycenter. If the person moves in a direction being orthogonal to this line, for instance, if the person moves in a longitudinal direction, a further measurement will lead to a position of the respective x-ray source being approximately located on another line being orthogonal to the detection surface. Generally, the detection of the positions of the x-ray sources at different times can lead to a complex movement pattern.

In an embodiment, the different times correspond to a temporally dependent physiological parameter like the respiratory cycle. In this case, simultaneously with acquiring the gamma camera images the respiratory cycle is measured by using, for instance, a corresponding respiratory belt.

The reconstruction unit 109 is adapted to use the determined motion, i.e. the positions of the x-ray sources determined at different times, for generating a motion corrected SPECT image. For performing this motion correction known motion correction algorithms can be used like one of the motion correction algorithms disclosed in the articles “Patient motion in thallium-201 myocardial SPECT imaging. An easily identified frequent source of artifactual defect” by J. Friedman et al., Clinical Nuclear Medicine, volume 13, issue 5, pages 321-324 (1988), “Detection and Correction of Patient Motion in Dynamic and Static Myocardial SPECT Using a Multi-Detector Camera” by G. Germano et al., The Journal of Nuclear Medicine, volume 34, pages 1349 to 1355 (1993) and “Performance of the automated motion correction program for the calculation of left ventricular volume and ejection fraction using quantitative gated SPECT software” by K. Uchiyama et al., Annals of Nuclear Medicine, volume 19, number 1, pages 9 to 15 (2005), which are herewith incorporated by reference. The function of such a motion correction algorithm will in the following be described by a simple example. The different gamma camera images acquired at different times can be regarded as being different frames. If, for instance, the person has moved 2 cm in the longitudinal direction between the fifth and the sixth frame, the barycenter of the respective one or several x-ray sources on the frames is shifted accordingly. The reconstruction unit then determines the 2-cm-movement from the shift of the barycenter of the one or several x-ray sources and considers this movement during the reconstruction of the SPECT image by, starting from the sixth frame, virtually shifting all nuclear detection events in the opposite longitudinal direction by 2 cm.

The x-ray sources 102 can be operated in different modes. For instance, each x-ray source 102 may be operated only for a short fraction of a frame.

The detection unit 106 with the at least one gamma camera, the reconstruction unit 109 and the x-ray sources 102 can be controlled by a control unit 111. In particular, the x-ray sources 102 can be controlled such that they are switched on when and for as long as needed for clearly identifying the x-ray sources in the acquired gamma camera image. For instance, the x-ray sources 102 can be controlled such that they emit the first radiation 105, when the gamma camera is relatively close to the respective x-ray source. In particular, the x-ray sources can be controlled such that they emit the first radiation, when the distance between an x-ray radiation source and a gamma camera is minimal or smaller than a predefined threshold of, for instance, 30 cm. Since the photon path of the first radiation between the x-ray sources 102 and the gamma camera is not obstructed by the person 3, photon attenuating and scattering effects can be avoided for the movement marker signal, i.e. attenuation and scattering effects do not adversely influence the detection of the x-ray source within the acquired gamma camera images.

Motions of the person 3 lead to a motion of the active markers, i.e. of the miniature x-ray sources 102. The shift of the marker positions caused by the motion of the markers can be extracted from subsequent marker measurements. The information about the marker motion is preferentially used to estimate the motion of the person 3 during SPECT acquisition, i.e. during the acquisition of the second, nuclear data 107 from the SPECT contrast agent 108.

The x-ray sources 102 can be controlled such that the discrimination of the first radiation against the second radiation and against the scatter background can be improved. For instance, the x-ray sources can be operated in accordance with temporal patterns, for instance, in order to allow the separation of signals of different x-ray sources from each other and/or from SPECT scatter background and to minimize additional dose burden for the person. Thus, also in this embodiment the x-ray sources can be adapted to generate x-ray radiation having an intensity being modulated in accordance with modulation characteristics, wherein the detection unit can be adapted to separate the first radiation from the second radiation based on the modulation characteristics. In particular, also the intensity of different x-ray sources can be modulated differently in accordance with different modulation characteristics, wherein the detection unit can be adapted to separate the first radiation from the different x-ray sources based on the different modulation characteristics. For instance, a lock-in technique or a Fourier transformation can be used, if the different x-ray sources are modulated with different modulation frequencies.

The distribution of the movement markers can be adapted to the SPECT acquisition scheme. For instance, if the gamma camera detects the radiation only over a certain angular range, the x-ray sources can be distributed such that they are visible, while detecting the radiation over this certain angular range. In particular, in cardiac SPECT nuclear radiation is generally detected over an angular range of 180 degrees only, wherein in this case the x-ray sources can be distributed such that the radiation from the x-ray sources can be detected by the gamma camera, if the gamma camera is moved within this angular range of 180 degrees. Moreover, if the expected motion has known main directions, for instance, if it is known that the motion is substantially respiratory motion having known certain main directions, the x-ray sources can be distributed such that movements in these main directions are very good detectable.

The x-ray sources 102 can, as already mentioned above, be operated in an energy range being different from the energy range of the second radiation, i.e. being different to the tracers emission line. Separate sets of projection data can therefore be obtained for the x-ray sources 102 and for the tracer substance, i.e. for the SPECT contrast agent 108, by applying corresponding energy windows to the projection data acquired by the detection unit 106. Alternatively or in addition, other techniques can be used for separating the detected first radiation from the detected second radiation. For instance, sub-frames with the x-ray sources switched on and off, i.e. temporarily consecutive images of the gamma camera, wherein in one image the x-ray sources are switched on and in another image the x-ray sources are switched off, can be subtracted from each other. If it can be assumed that between the acquisition of these images, i.e. of these sub-frames, the positions of the person and the gamma camera have substantially not been modified, the parts of these images, which do not correspond to the x-ray sources, have also not been modified such that the resulting subtraction image mainly shows the x-ray sources, thereby separating the detected first radiation from the detected second radiation.

Since the reconstruction unit 109 can be adapted to perform a tomographic reconstruction for determining the center positions of the approximately point-like x-ray sources 102, the center positions of the x-ray sources 102 can be estimated from few projections, i.e. by the first radiation detected in few different directions, at a relatively high spatial precision, in particular, at a spatial precision being higher than that achieved in the SPECT detection of the distribution of the SPECT contrast agent 108 inside the person 3.

Typical acquisition times in SPECT imaging are in the order of half an hour. Patient motion during this period of time can severely deteriorate the achievable image quality. The SPECT imaging system 101 described above with reference to FIG. 3 corrects the SPECT data and, thus, restores a high SPECT image quality. Since the detection unit 106 of the SPECT imaging system 101 detects both, the first radiation 105 generated by the x-ray sources 102 and the second radiation 107 caused by the SPECT contrast agent 108, the corresponding projection data are inherently registered with respect to each other. A further motion tracking system, for instance, a separate optical motion tracking system, is not necessarily required. Moreover, by using the x-ray sources 102 as active markers for detecting the motion instead of, for instance, radioactive pellets, the markers can be switched on just when and for as long as necessary for determining the motion of the person, even in complex temporal patterns. These switching procedures can be used to differentiate projection data, which correspond to the first radiation, from projection data, which correspond to the second radiation. Moreover, the switching can reduce the dose applied to the person 3 to a minimum. Also the ability to individually adjust the acceleration voltage resulting in x-ray energies well below the emission energy of the applied SPECT tracer, i.e. of the SPECT contrast agent 108, in combination with multi-energy window acquisition makes the differentiation between the detected first radiation and the detected second radiation relatively easy. Moreover, since the x-ray sources can be operated in relatively short time intervals with a relatively high intensity changes in patient position can be determined relatively fast and accurately.

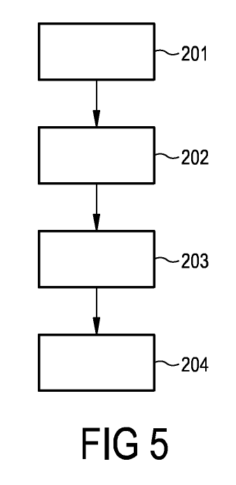

In the following an embodiment of a nuclear imaging method for imaging an object in an examination region will exemplarily be described with reference to a flowchart shown in FIG. 5.

In step 201, first radiation being x-ray radiation is generated by multiple x-ray sources, wherein the x-ray sources are arranged such that the detected x-ray radiation is indicative of a property of an object. For instance, the x-ray sources can be arranged along a part of a ring or along a full ring surrounding the person and being axially offset to or being integrated within a PET detector ring, wherein the x-ray sources are operated such that the first radiation transmits the person in different directions as described above with reference to FIG. 1. In this case, the first radiation is indicative of the attenuation of the person. Alternatively, the x-ray sources can be attached to the outer surface of the person 3 such that the x-ray sources move with movements of the person 3 as described above with reference to FIG. 3. The detected x-ray radiation is then indicative of a movement of the person 3, i.e. of the positions of the person 3 at different times.

In step 202, a detection unit detects second radiation from a nuclear element, after the radiation has traversed the person, and the first radiation generated by the multiple x-ray sources. For instance, the detection unit can comprise a PET detector ring, which detects radiation from a PET contrast agent and the first radiation generated by x-ray sources arranged on a part of a ring or a full ring surrounding the person. Alternatively, the detection unit can comprise one or more gamma cameras being adapted to detect radiation of a SPECT contrast agent administered to the person and first radiation from x-ray sources attached to the person.

In step 203, a corrected nuclear image of the person is reconstructed based on the detected first radiation and the detected second radiation by a reconstruction unit, wherein the nuclear image is corrected with respect to the property of the object. For instance, the reconstruction unit can be adapted to generate an attenuation map based on the first radiation and to reconstruct an attenuation-corrected PET image of the person based on acquired PET data being the detected second radiation and based on the attenuation map. Alternatively, the reconstruction unit can be adapted to determine the motion of the person based on detected first radiation from x-ray sources attached to the person and to use the determined motion for reconstructing a motion-corrected SPECT image of the person.

In step 204, the reconstructed corrected nuclear image is shown on a display unit.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Calculations like the reconstruction procedures and the correction procedures performed by one or several units or devices can be performed by any other number of units or devices. For example, step 203 can be performed by a single unit or by any other number of different units. The calculations and/or the control of the nuclear imaging system in accordance with the nuclear imaging method can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

The invention relates to a nuclear imaging system for imaging an object in an examination region. Multiple x-rays sources generate first radiation being x-ray radiation, wherein the x-ray sources are arranged such that the x-ray radiation is indicative of a property of the object. A detection unit detects second radiation from a nuclear element, after the second radiation has the traversed the object, and the first radiation generated by the multiple x-ray sources, thereby inherently registering the detection of the first radiation and the second radiation. A reconstruction unit reconstructs a corrected nuclear image of the object based on the detected first radiation and the detected second radiation, wherein the nuclear image is corrected with respect to the property of the object and, because of the inherent registration, does not comprise image artifacts caused by registration errors.

1. A nuclear imaging system for imaging an object in an examination region, wherein the nuclear imaging system comprises:
multiple x-rays sources for generating first radiation being x-ray radiation, the x-ray sources being arrangable such that the x-ray radiation is indicative of a property of the object,
a detection unit, and
a reconstruction unit,
wherein the detection unit is configured to detect second radiation from a nuclear element, after the second radiation has the traversed the object, and the first radiation generated by the multiple x-ray sources, and in that
the reconstruction unit is configured to reconstruct a corrected nuclear image of the object based on the detected first radiation and the detected second radiation, wherein the nuclear image is corrected with respect to the property of the object.

2. The nuclear imaging system as defined in claim 1, wherein the multiple x-ray sources are arranged to allow the generated x-ray radiation to traverse the object such that the x-ray radiation is indicative of the absorption of the object.

3. The nuclear imaging system as defined in claim 2, wherein the multiple x-ray sources are arranged around the examination region for generating first radiation traversing the object in different directions and wherein the detection unit is adapted for detecting the first radiation having traversed the object in different directions.

4. The nuclear imaging system as defined in claim 3, wherein the reconstruction unit is adapted to reconstruct an attenuation image of the object being indicative of the absorption distribution within the object based on the detected first radiation and to generate an attenuation corrected nuclear image based on the detected second radiation and the reconstructed attenuation image.

5. The nuclear imaging system as defined in claim 4, wherein the nuclear element is a nuclear position emission tomography (PET) contrast agent, wherein the detection unit comprises a detector ring surrounding the examination region for detecting the second radiation in different directions, wherein the reconstruction unit is adapted to reconstruct an attenuation corrected PET image based on the detected second radiation and the attenuation image.

6. The nuclear imagine system as defined in claim 1, wherein the multiple x-ray sources are adapted to be arranged on the object such that the x-ray radiation is indicative of a movement of the object.

7. The nuclear imaging system as defined in claim 6, wherein the reconstruction unit is adapted to determine the movement of the object based on the detected first radiation and to reconstruct a motion corrected nuclear image based on the detected second radiation and the determined movement of the object.

8. The nuclear imaging system as defined in claim 7, wherein the nuclear element is a nuclear single photon emission tomography (SPECT) contrast agent, wherein the detection unit comprises at least one gamma camera being adapted to detect the second radiation in different directions and to detect the first radiation, wherein the reconstruction unit is adapted to reconstruct a motion corrected SPECT image based on the second radiation detected in different directions and the determined movement of the object.

9. The nuclear imaging system as defined in claim 8, wherein the at least one gamma camera is adapted to detect also the first radiation in different directions, wherein the reconstruction unit is adapted to determine the positions of the multiple x-ray sources over time from the first radiation detected in different directions, thereby determining the movement of the object.

10. The nuclear imaging system as defined in claim 1, wherein the x-rays sources are adapted to be activated in a predefined temporal pattern and wherein the detection unit is adapted to detect the first radiation based on the predefined temporal pattern.

11. The nuclear imaging system as defined in claim 1, wherein the x-ray sources are adapted to generate x-ray radiation having an intensity being modulated in accordance with modulation characteristics and wherein the detection unit is adapted to separate the first radiation from the second radiation based on the modulation characteristics.

12. The nuclear imaging system as defined in claim 11, wherein the intensity of different x-ray sources is modulated differently in accordance with different modulation characteristics and wherein the detection unit is adapted to separate the first radiation from the different x-ray sources based on the different modulation characteristics.

13. The nuclear imaging system as defined in claim 1, wherein the detection unit is adapted to detect the first radiation in a first energy range and the second radiation in a second energy range.

14. A nuclear imaging method for imaging an object in an examination region, wherein the nuclear imaging method comprises:
generating first radiation being x-ray radiation by multiple x-ray sources, the x-ray sources being arrangable such that the x-ray radiation is indicative of a property of the object,
detecting, and
reconstructing a corrected nuclear image of the object,
wherein detecting comprises detecting second radiation from a nuclear element by a detection unit, after the radiation has the traversed the object, and the first radiation generated by the multiple x-ray sources, and in that
reconstructing a corrected nuclear image of the object comprises reconstructing a corrected nuclear image of the object based on the detected first radiation and the detected second radiation by a reconstruction unit, wherein the nuclear image is corrected with respect to the property of the object.

15. A nuclear imaging computer program for imaging an object, the nuclear imaging computer program comprising program code means for causing a nuclear imaging system to carry out the steps of the nuclear imaging method as defined in claim 14, when the nuclear imaging computer program is run on a computer controlling the nuclear imaging system.
